(19) United States
(12) Patent Application Publication
(10) Pub. No.: US 2024/0252573 A1
(43) Pub. Date: Aug. 1, 2024

(54) KINGLAIT SPORT DRINKS' LINE DESCRIPTION & METHODS OF MANUFACTURING

(71) Applicant: Aladdin Anwar Ghanem, Simi Valley, CA (US)

(72) Inventor: Aladdin Anwar Ghanem, Simi Valley, CA (US)

(21) Appl. No.: 18/161,055

(22) Filed: Jan. 28, 2023

(51) Int. Cl. A61K 36/235; A61K 33/00; A61K 33/04; A61K 33/06; A61K 33/22; A61K 33/24; A61K 33/26; A61K 33/30; A61K 33/32; A61K 33/34; A61K 35/20; A61K 38/01; A61K 47/12; A61K 47/28; A61K 47/36

(52) U.S. Cl. CPC A61K 36/235; A61K 33/00; A61K 33/04; A61K 33/06; A61K 33/22; A61K 33/24; A61K 33/26; A61K 33/30; A61K 33/32; A61K 33/34; A61K 35/20; A61K 38/018; A61K 47/12; A61K 47/28; A61K 47/36

(57) ABSTRACT

Customary sweetened healthy line of sport drinks. Utility and methods of manufacturing are disclosed. The products' line is designed to benefit healthy consumers beginning of age 5 Years old and up. The liquid drinks include sterilized milk or Lactaid 100%, organic or natural type derived from grass fed cows given no growth hormones or antibiotic and rich in Omega-3 and conjugated linoleic acid (CLA). The milk is mixed with a portion of water having the pH between 6.5 and 8.5. Each sport drink further includes water soluble dietary fiber and a bouquet of proprietary essential oils or botanical extracts blend known for its natural benefits against many human ailments and tend to maintain the health of blood, kidney and digestive tract. The products' liquid solutions exhibit an average specific gravity range between 0.75 to 1.150. Additional ingredients such as natural preservatives, emulsifiers, proteins, vitamins and minerals may also be included to further enhance the aesthetics or authenticity of the presented line of intended healthy drinks. The manufacturing methods are simple but articulate.

### FIELD

This invention relates to the field of sport drinks. More particularly, this invention relates to new formulations of sport drinks and methods for making them.

### INTRODUCTION

Sport drinks are beneficial for hydrating and replenishing spent substances in the human body.

### SUMMARY

According to one aspect of the disclosure there is described a sport drink including the ingredients of water, preservative, mineral, trace mineral, dietary fiber, milk, sweetener, essential oil, emulsifier, at least 18% dairy fat, rheology modifier, vitamin, and protein.

In some embodiments according to this aspect of the disclosure, the weight percent of the ingredients includes water from 5% to 25%, preservative from 0.01% to 0.19%, mineral from 0.01% to 0.09%, trace mineral from 0.001% to 0.009%, dietary fiber from 0.5% to 3.5%, milk from 55% to 95%, sweetener from 2% to 9%, essential oil from 0.15% to 0.55%, emulsifier from 0.5% to 1.5% rheology modifier from 0.25% to 1.75% vitamin from 0.05% to 0.09%, and protein from 0.1% to 1.9%.

In some embodiments, a ratio of water to milk is from 1:2.2 to 1:19. In some embodiments, a weight percent of the essential oil is from 0.10% to 0.60%. In some embodiments, a weight percent of sweet fennel oil in the essential oil is from 10% to 80%. In some embodiments, a weight percent of sucrose in the sweetener is from 5% to 75%. In some embodiments, the essential oil has antimicrobial properties. In some embodiments, the preservative comprises at least one of sodium benzoate and potassium sorbate, and a weight percent of the preservative to essential oil is from 5% to 75%. In some embodiments, a weight percent of polysorbate in the emulsifier is no more than 35%. In some embodiments, a weight percent of at least one of casein and whey in the protein is from 1% to 95%. In some embodiments, a concentration of any one trace mineral in the sport drink is from 10 ppm to 100 ppm, a concentration of all the trace mineral in the sport rink is no more than 1000 ppm. In some embodiments, a weight percent of arrow root starch thickener in the rheology modifier is from 1% to 95%.

### DETAILED DESCRIPTION

In a human body, the balance between acidity and alkalinity is referred to as “Acid Base Balance” or sometimes called “Acid-Base Equilibrium”. An important property of blood is its level of acidity. or alkalinity. That is expressed by the hydrogen ion concentration or pH value. Under normal conditions, human blood is slightly basic with a pH range of 7.35 to 7.45. However, the body often tends to precisely maintains the pH of blood close to 7.40. Any deviation from the normal range can severely affect the function of many body organs.

Studies reveal that blood acidity increases when level of acidic compounds in the body rises through increased intake, production or decreased elimination. On the other hand, the level of basic (alkaline) compounds falls through decreased intake, production or increased elimination.

Acidosis and Alkalosis are two abnormal conditions of the blood where acidosis is when the blood has too much acidity while alkalosis is when the blood has too much alkalinity. The human body uses different mechanisms to precisely control the pH level of the blood. These mechanisms involve Lung, Kidneys and buffer systems. The lung releases carbon dioxide (CO2), a mildly acidic compound, which is a waste product of the metabolism of oxygen and a variety of nutrients. Carbon dioxide is carried by blood to the lung to be exhaled. The brain regulates the amount of CO2 exhaled by controlling the speed and depth of breathing (ongoing body ventilation) hence, is able to regulate the pH of the blood minute by minute. Kidneys have the ability to alter the pH of the blood by releasing acidic or basic material and by controlling their amount.

The third mechanism of controlling the pH level is through chemical buffer systems. Said system guards against sudden shifts in acidity or alkalinity. The pH buffer systems are combinations of the body's own naturally occurring weak acids and weak bases. The most important buffer system involves a ratio between carbonic acid, formed by Carbon dioxide dissolved in the blood, and the bicarbonate ion which is the corresponding weak base.

During blood circulation, the metabolic reactions are at their best due to an optimum condition of acid base equilibrium. This is translated into healthy digestive tract, the release of enough energy and efficient detoxification process. Drinking soft drinks of high sugar and no fibers, is possibly associated with obesity, type-2 diabetes and weight gain. However, sodas can also have ill effects on the bones and may potentially lead to visible teeth cavities or decay. The sugars of the acidic carbonated drinks interact with bacteria in the mouth to release more acids. Kidney function was found to decline over two decades in women who drank several diet sodas a day.

The theory is that Phosphoric acid, which is added to enhance flavors in some carbonated drinks, can interfere with calcium absorption and leads to some loss of calcium from the blood. Thus, the consumption of too much of said acidified drinks has been linked to some drop in kidney functions and the possible formation of kidney stones. For this reason, our line of products is concerned with the development of healthy sport drinks featured with the proper blood pH supportive ingredients, kidneys' protective dietary fiber and material to prevent the loss of calcium. Thus, the products aim to diminishes the risk of osteoporosis and other associated ailments. Such featured drinks are expected to be at high demand.

This disclosure relates to the composition, utility and manufacturing procedures of a sweetened healthy line of affordable sport drinks. The drinks involve the use of natural dietary fiber dissolved in natural or organic non-GMO, antibiotic free, sterilized milk mixed with water that is loaded with selected essential oil blends and other nutritive ingredients. The overall structure of the products' line is designed to improve or maintain the quality of human health through targeting three body organs: Blood, kidney and the digestive tract. The target audience of said embodiments shall be all genders of healthy children age 5 and up, youths, adult persons, elderly consumers, athletes, dairy industry management and nutritional research specialists of all geographic, psychographic, behavioral and demographic areas.

The disclosure is inspired by the fact that healthy blood, kidney and digestive tract are the main organs behind healthy life. Many studies, conducted before, confirmed that diet diseases, or fevers, first start at the digestive tract followed by blood illness then malfunction of kidney. Therefore, the ingredients selected for this embodiment of sport drinks are carefully selected to nourish and support the function of all of these body organs. Furthermore, prone to help in the daily detoxification process.

However, this embodiment is also intended to fill the gap between what we should consume on daily basis, for better health, and what is missing in the beverages we consume. Many research specialists have attempted to address our health issues. While some succeeded in identifying the root cause, very few translated their works into useful products. Thus, the scope of the embodiment is about formulating a line of drinks that are able to help improve human health. Our endeavor, therefore, involves an ongoing search to formulate drinks that are statutory, novel, useful and nonobvious.

While milk does more than building strong bones, its diverse Poquette of vitamins and minerals can be a valued nutrition powerhouse. Many studies show clear association between dairy intake and reduced risk of Osteoporosis. The blood maintains a level of Calcium within a narrow range. If you are not getting Calcium from the blood, the body will get it from the bones. Even though there are so many food sources for Calcium, milk is still the best source for soluble and deliverable Calcium for all ages.

Research shows that milk from grass fed cows raised on pasture may have up to 92% more Omega-3 fatty acids and 94% more conjugated linoleic acid (CLA) than milk from conventionally raised cows. The presence of natural dietary fibers side by side with milk will normalize bowel movement, soften and increase the weight and size of the stool while delivering vital nutrition. Furthermore, fibers lower the LDL, (low density lipoprotein bad cholesterol), help control blood sugar level and tends to participate in the prevention of intestinal cancer.

Gum Arabic extracts protect against hepatic oxidative stress and onset of STZ induced diabetic kidney disease. Gum Arabic extracts protect against hepatic oxidative stress in alloxan induced diabetes in rats.

Dietary fibers do not get digested, hence, do not turn into sugars or glucose in the body. Even though dietary fibers are included in the total carbohydrate count, yet it does not absorb like other carbohydrate. Thus, it shouldn't count in the overall intake of carbohydrate for “what doesn't absorb doesn't count”. While the proper daily intake of fibers help regulates the digestive process, it may also facilitate the absorption of more nutrients from the Chymus through increasing the surface area of absorption. Chymus is a final murky semifluid mass or paste containing variety of nutrition in a water-soluble form. Thus, ingesting fibers won't block the ability of the body to absorb vitamins and minerals.

Each member of the Essential oils' list selected for the invented products' line has been identified by research specialist as natural remedy when used alone or in a blend with other members. The research personnel designated each oil to be a booster to the immune system or an antimicrobial growth or acting as natural preservative, antiseptic or antispasmodic or aperitif or carminative or anti-inflammatory. Some oils exhibit more than one of the previous functions. Additional details about the specific features of some oils will be disclosed in this disclosure. Enhancing the formulae with additives such as natural preservatives, emulsifiers, vitamins, and minerals shall extend the shelf life and increase the nutritional value, aesthetics or authenticity of the embodiment.

This disclosure is about formulating, blending and packaging of affordable nonalcoholic healthy line of sport drinks. The formulae ingredients are all-natural and of health remedies beginning with the watered non-GMO milk (whole or skimmed), the soluble dietary fibers, the organic or natural sugars and the essential oils or their blends. Other natural FDA approved food ingredients are added to enhance and preserve the ayurvedic and authenticity of the products' line. Such additives are preservatives, emulsifiers, dairy fats, proteins, vitamins, rheology modifiers, minerals and trace minerals.

The idea of the line was conceived after careful observation about the USA market and confirming that it is lacking healthy drinks. Medical reports stating that healthy blood, efficient kidney and digestive tract are the main organs behind holistic wellbeing of human health directed the research focus to be on identifying novel ingredients to formulate the products.

Therefore, one goal is to introduce drinks that are natural remedies to optimize the chemistry of blood, boost the function of kidneys and to improve the environment around the digestive tract. Furthermore, one objective is to present simple unambiguous cost-efficient manufacturing procedures about a product line made with easily available ingredients.

The target audience of said disclosure shall be all genders of healthy children beginning of age 5, youth, adult persons, elderly consumers, Athletes, dairy industry managers and nutritional research specialists of all geographic, psychographic, behavioral and demographic areas. The products' line involves healthy drinks of a pH varies between 6.0-8.5 and a specific gravity range from 0.75 and 1.15 gm/cm3. Accordingly, the general composition of the products' line, ingredients and manufacturing procedures are claimed.

It is customary sweetened healthy line of sport drinks targeting wide range of audience of all genders healthy consumers beginning of children age 5 and up in all geographic, psychographic, behavioral and demographic areas. The elements of this embodiment include: water, milk, dietary fiber, proprietary blends of specialty essential oils, natural preservatives, dairy fat, food emulsifiers, vitamins, proteins, rheology modifiers, minerals and trace minerals.

One purpose of this disclosure is to introduce a line of natural healthy drinks to support the optimum chemical structure of the blood, the function of the kidney and to idealize the environment inside the digestive tract. The detailed description of each categorized ingredient used in making the drinks, proprietary essential oil blends, the natural dietary fibers, additives and the types of milk as well as the methods of manufacturing shall be described herein in detail.

### Essential Oils

Essential oils selected for formulation are used individually or as part of proprietary blend of two to five members from the list. When blended according to the designated ratio, they are loaded on dairy fats or suitable emulsifier before addition to the batch. Essential oils are concentrated hydrophobic compounds, tending to repel water or fail to mix with it. They are liquids containing volatile compounds which are easily evaporated at normal temperature. They are extracted from plants, mostly by steam distillation or solvent extraction. Said essential oils are often used for aromatherapy or as alternative medicine.

One focus of this disclosure is on the use of edible oils and their intrinsic medicinal benefits. The list includes: Sweet Fennel seed oil, Star anis oil, Lavender oil, Cardamum oil, Nutmeg oil, Cinnamon oil, Peppermint oil, Rose oil, Rosemary oil, Clary sage oil, Frankincense oil, White fir oil, Pomegranate seed oil, Rosehip oil, Basil oil, Black seed oil, Cumin seed oil, Cannabis CBD oil, Hemp seed oil, Flax seed oil, White thyme oil, White fir oil, Orange blossom oil, Celery oil, Cloves oil, Dill oil, Radish oil, Sesame seed oil, Grape seed oil, Chamomile oil, Pumkin oil, Ginger oil, Black pepper oil, Capsicum oil Garlic oil, Mustard seed oil, Oregano oil, Turmeric oil, Onion oil, Arugula oil, Dandelion oil, Argan oil, Moringa oil, Watercress seed oil, Carrot oil, Wheat germ oil, Rice bran oil, Tea tree oil, Japanese star oil, Maca oil, Bergamot oil, Lemongrass oil, Jasmine oil, Lavender oil and Ylang ylang oil. Sweet Fennel, Foeniculum vulgare dulce, and Star anis, Illicium verum, oils are the first members selected for the formulae development.

They may exist as basic oils in formulating most of the entire line. Fennel dry seeds and extracted oil has history of being used by ancient Egyptians in food and as medicine. It is thought to be a remedy for snake bites toxicity. Fennel seed and its oil extract have many applications in the culinary industry such as baking, ice cream, condiments, salads and teas. Sweet fennel seed and Star anis oils' proprietary dual blend, alone or in a blend with other oils, tend to introduce unique flavors and anoint the body with plenty of benefits. When taken internally, at low concentrations, sweet fennel or star anise oil reduces or prevents spasms in the gut, demonstrates antioxidant properties, antimicrobial characteristics, relieves gas, constipation and aids in weight loss.

They also demonstrate antimicrobial benefits to the gum that is enhanced with the addition of cloves oil. Colic, (an intense baby crying in the first three months for no clear reason, happens as an expression of pain and often occurs at night) is a problem in infants which imparts a significant emotional and physical burden to parents. While Dicyclomine hydrochloride is widely used pharmacological treatment for infantile's colic; unfortunately, 5% of the infant treated with said medicine developed serious side effect leading to death in some cases.

Sweet fennel seed and star anise oils introduced alone or blended with Caraway oil has shown significant decrease in the infants' Colic symptoms, thus prepare the babies for a good night sleep. Many studies suggests that Fennel seed oil emulsion is superior in decreasing intensity of infantile colic. The oils' blend was found to demonstrates antifungal activity against Candida albicans which is a fungus like organisms live with other microflora members in the intestine believed to cause symptoms such as fatigue, headache and poor memory.

The benefits of the sweet fennel and star anis oils is attributed to properties such as Antiseptic, suppresses or slows down the growth of disease causing microorganisms, Antispasmodic, relieves spam of involuntary muscles, Aperitif, simulates the effect of an alcoholic drink taken before a meal to stimulate the appetite, Carminative, relieving flatulence or the accumulation of gas in the alimentary canal from mouth to anus including esophagus, stomach & intestine and finally as Anti-inflammatory, a substance that reduces inflammation illustrated by redness, swelling and pain.

Fennel seed oil, by itself, has 28 identified compounds mainly Trans-anethole, a phenylpropanoid and Estragole, a phenylpropene, also an isomer of anethol. In addition, the oil has acetone, n-butanol and ethanol. Trans anethole is a chief component of sweet fennel seed oil and several essential oils including star anise. Some substances of this class exhibit local anesthetics where they block excitability predominately through direct inhibitory effect on Na+, sodium ion channel conductance activation. Said compounds work synergistically to inhibit the growth of wide spectrum of bacteria and fungi, hence act as preservatives besides their pleasant flavor. Trans anethole is a Chief component of several essential oils including star anise seed oil.

However, limitations of Fennel dry seeds or oil extract, in particular, suggest that it shouldn't be used at all or in excessive amounts by consumers who are allergic to Fennel or have past history or present one of cancer of the Breast, Ovary or Uterus or a history of Endometriosis, a disorder in which the tissue that normally lines the uterus grows outside the uterus or Uterine fibroids, also called Uterine myoma, a noncancerous growth in the uterus that can develop during a woman's childbearing years. Ingesting sweet fennel oil or eating the seeds might increase the risk of bleeding or bruising in people with bleeding disorder. Hormone sensitivity conditions such as in Breast, Uterine or Ovarian cancer, Endometriosis or Uterine fibroids where Fennel might act like estrogen. Food poisoning and spoilage caused by microorganisms are still the most important issue facing the food industry and consumers.

To avoid contamination and consequences of the growth of harmful microbes, the food industry has used synthetic additives to diminish microbial growth and inhibit microorganisms. However, because of the consumers' growing concerns over the safety of foods containing synthetic chemicals, a lot of attention has been paid to the use of natural antimicrobial products as food preservatives. Thus, spices and their essential oil extracts have received attention to their useful physiological function and antimicrobial activities.

However, in addition to all remedies and flavors intrinsic to said essential oils, they can act as preservatives or microbial growth inhibitors. Sweet Fennel and Star anis are perennial herbs. They are widely cultivated throughout temperate and tropical regions of the world. Not only used in culinary recipes such as bread, pickles, pastries and cheese but also as flavoring reagents in liqueurs. Reports on the relative oil yields greatly depends on the geographical area of the plant origin, extraction methods, maturation stages and the designated part of the plant used for extraction. The extracts are also used as a constituent in cosmetics and pharmaceutical products.

To maintain a good quality of this invented line of sport drinks, all of the quality differences of the oil must be taken into consideration. Various reports about biological activities of the essential oils such as Hepatoprotective effect, antioxidant activities, anti-bacterial activity, anti-inflammatory activity, antithrombotic activity which is a function of two classes: anticoagulants & antiplatelets drugs.

Anticoagulant slows down clotting thereby reducing fibrin formation, hence, preventing clots from forming or growing. Antiplatelets agent ‘prevents platelets from clumping, hence prevents clots from forming, antidiabetic activity, anti-tumours activity acaricidal activity, a pesticide designed to control harmful species of mites. Repeated use of these acaricides has resulted in the resistance and undesirable effect on non-target organisms and also fosters serious human health concerns. Plant extract may be an alternative source for chemical control of dust mites.

However, information on the antimicrobial mechanism against microbes of the essential oils from fennel and other members in the list still limited and under observation. The chemical composition of the oils is obtained by Hydro-distillation of air-dried samples. In the case of Fennel, the yield is 1.74% (v/w). The compositions of Fennel oil were separated by GC-Gas chromatography and identified by GC-MS, gas chromatography/mass spectrophotometry. The results identified 28 different compounds representing 95.9% of the total sample amount. Trans anethole 68.53%, a phenylpropanoid as the main component, Estragole 10.24% was the second major phenylpropene compound detected followed by Limonene 6.24%.

Based on many research work, the essential oil from Fennel seed was found rich in Trans anethole Antiplatelets agent prevents platelets from clumping, hence prevents clots from forming, antidiabetic activity, anti-tumours activity, acaricidal activity, a pesticide designed to control harmful species of mites. Repeated use of these acaricides has resulted in the resistance and undesirable effect on non-target organisms and also fosters serious human health concerns. Plant extract may be an alternative source for chemical control of dust mites. However, information on the antimicrobial mechanism against microbes of the essential oils from fennel and other members in the list still limited and under observation.

The chemical composition of the oils is obtained by Hydro-distillation of airdried samples. In the case of Fennel, the yield is 1.74% (v/w). The compositions of Fennel oil were separated by GC-Gas chromatography and identified by GC-MS, gas chromatography/mass spectrophotometry. The results identified 28 different compounds representing 95.9% of the total sample amount. Trans anethole 68.53%, a phenylpropanoid as the main component, Estragole 10.24% was the second major phenylpropene compound detected followed by Limonene 6.24%. Based on many separated by GC-Gas chromatography and identified by GCMS, gas chromatography/mass spectrophotometry.

Based on research work, the essential oil from Fennel seed was found rich in Trans anethole possessing good antibacterial activity against food borne pathogens. It was concluded that the mechanism of action of the oil can work against Shigella dysenteriae, a gram negative, facultative anaerobic, non-spore forming, nonmotile, rod-shaped bacteria and genetically closely related to Escherichia Coli bacteria. Shigella species can cause shigellosis of the symptoms fever, bloody diarrhea, severe stomach cramping or tenderness or sensitivity to pain, and dehydration.

Star Anise is a medium sized evergreen tree native to Egypt, North Africa, Eastern Mediterranean region, Southern Europe, southern Russia, Northeast Vietnam, southwest China, Pakistan, Chile, Mexico and the United States. A spice commonly known as star anise, Star anise, Star anise seed, star aniseed, star of anise or Chinese star. It is a member of the Schisandraceae family, a family of flowering plants with three known genera and a total of 92 known species. Star anise essential oil has an aroma similar to black licorice.

The major components of star anise essential oil identified by GC/MS were Trans-anethole 82.7%, Carryophyllene 4.8%, limonene 2.3%, Estragole 1.14, Anisaldehyde 0.94%, Ethanol and Limonene. Chinese star anise contains 5% to 7% Shikimic acid while pine needles only contain 1.5%. Star anise oil composition makes it compatible candidate to blend with Fennel seed oil. The chemical composition of Star anise, to great extent, is similar to Fennel seed oil. Star anise exhibits anti-fungal, antibacterial and anti-inflammatory properties.

Prone to fight stomach ulcers, keeps blood sugar levels in check and reduces symptoms of depression and menopause. Combined with nutritious diet and healthy lifestyle, Star anise oil could improve several aspects of human health. Star anise has sedative properties that can help decrease anxiety and to settle down nerves for good night sleep when drinking a cup of star anise tea before bed.

Normal metabolism can produce free radicals that can damage cells if they are in excessive level. Under normal conditions, reactive oxygen species (ROS) are eliminated by the body antioxidants including the nonenzymatic components and antioxidant enzymes. High levels of ROS have been shown to disturb oxidation, antioxidant defense and leads to lipid peroxidation to cause oxidative damages to proteins and DNA. This issue imposes negative impact on the productive performance of birds and leads to an oxidative stress.

However, the use of synthetic antioxidants has been banned due to their toxicity. Instead, growing interest in using Phytogenic feed additives is increasing to attain high antioxidant status which has been regarded as one of the major factors positively affecting birds' performance. Star anise seeds and its essential oils are generally recognized as safe (GRAS) to achieve the goal and has been used widely in food, brewery and in health supplement industry.

### Milk

Milk, as designated, is a major ingredient of this embodiment, known as an excellent source of vitamins and minerals such as: Vitamin A & D, Potassium, Calcium, Selenium, Magnesium and Zinc. These ingredients are among 250 useful chemical compounds identified in milk. Other useful components are water (average 87%), fat, Protein (Casein & Whey), Carbohydrate Lactose) and other minerals.

There are numerous highly important micronutrients that exist in milk, such as essential amino acids and trace minerals. The combination of Calcium and Phosphorous makes milk necessary for building and maintaining strong bones and teeth. Calcium, Phosphorous and Vitamin D are three ingredients out of four the USDA deems under consumed by most American.

The Physical properties of milk: density, viscosity, freezing point, optical feature and acid base equilibria suggested that milk is the proper candidate to house the component of this sport drink line. Whey and Casein proteins are the major proteins in milk. They serve as a source of energy and help building and repairing muscle tissue.

In addition, milk contains riboflavin (B2), Niacin (B3), Pantothenic acid (B5) and Cobalamin (B12) besides Iodine and Selenium. The milk used in this embodiment is non-GMO, natural or organic, skimmed or non-skimmed, obtained from grass fed animals free from hormones or antibiotics. The primary type of milk is whole milk. The gross composition of cow's milk in the US is 87.7% water, 4.9% Lactose (Carbohydrates), 3.4% fat, 3.3 proteins and 0.7% minerals, referred to as ash. The composition of milk continuously undergoes changes depending on breeding, feeding strategies, type and management of the animal, lactation stage and season.

Reduced fat milk is 2% fat while skim milk is about 1% fat while fat free milk contains 0.1% to 0.3% milk fat. The milk used in this embodiment is sterilized at ultra-high temperature (UTH) where all forms of life has been removed, killed or deactivated through rapid heating to 135 C followed by quick cooling. There is no significant difference in taste or in nutritive value between UTH treated milk and regular one. UTH method kills E. Coli and Salmonella as well as other nonpathogenic bacteria that can cause spoilage. However, UTH milk, sometimes called Long-Life milk, has a typical shelf life of 6 to 9 months at ambient temperatures if unopened. Once opened, it should be refrigerated and used within 7 days.

The addition of essential oils' extract enhances the shelf life of the sport drink line.

### Water

Water or aqua, used in the manufacturing of this sport drink line is non-carbonated i.e., without any bubbles. It shall be purified spring or main water. When main water is used it must undergo further treatment before it is used for production. The pH of the employed water should be between 6.0 and 9.0. Spring or mains water can be used if it meets the pH and sterilization criteria. Traces of K+, Na+, Mg++, and Ca++ in part per million level shall be allowed.

Water exposed to UV light is permissible. UV light is normally effective against many Viruses, Bacteria and Protozoa. However, some microorganisms such as Cryptosporidium and Giardia have thick protective or cell walls that some low power of UV light systems is not able to penetrate. The proper dose of UV light is efficient in killing Bacteria within 10 seconds. It is able to destroy 99.99% of harmful microorganisms. Among the methods accepted for purifying the water is reverse osmosis.

In all cases, nanofiltration might be required depending on the quality of the source.

### Dietary Fiber

Natural Dietary fiber: Gum Arabic, also known as Acacia Senegal, Gum Sudani, Acacia Gum or Gum Acacia, Arabic Gum, Acacia Senegal gum or Indian Gum. Gum Arabic is a natural gum consisting of the hardened sap exuded of two species of the Acacia Sensu Lato tree: Senegalia senegal and Vachellia seyal. The substance is also used in making safe adhesives, pharmaceuticals, inks, confections, and other products.

Gum Arabic is a complex of glycoproteins and polysaccharide predominately polymers of arabinose and galactose. It is indigestible for both humans and animals. This Acacia Gum contains water soluble dietary fibers (WSDF) and has been considered safe for use in food and cosmetics by the United States FDA since 1970. It will be dissolved in hot and cold water through medium agitation until clear solution. However, it may not be heated to boiling degree for it will change color and adhesive properties.

Gum Arabic, in food or in drinks, improves patients' digestive systems and appetite. It is a good remedy for healthy consumers and patients of sick kidneys. It helps consumers to get enough energy from their food and in keeping cholesterol under control. It has anti-inflammatory activity through its derivative butyrate. Gum Arabic is a probiotic since it stimulates the growth and activity of beneficial bacteria of the intestinal flora. It is one of the best studied of the nonabsorbable carbohydrates.

In 1969, Gum Arabic was evaluated and accepted for human daily intake by FAOWHO joint expert committee on food additives. However, Sudanese people has been using it in the western Sudan in diets and drinks without limitations. It does not get degraded in the intestine since it is indigestible but ferments in the colon to give short chain fatty acids leading to an impressive range of health benefits. It has been claimed that four weeks supplementation of 10 grams per day leads to significant increase in Bifidobacteria, Lactobacteria and Bacteroides; indicating positive probiotic properties.

Many epidemiological studies suggest that ongoing intake of dietary fibers such as Gum Arabic is associated with beneficial effect on fat metabolism. Dietary fibers promote satiation and satiety, alter glycemic index, affect gastric emptying, gut hormones secretion and thus helps manage body weight.

Leptin hormone, a mediator of long-term regulation of energy balance, suppresses food intake and thereby inducing weight loss. A study showed that Gum Arabic inhibits intestinal glucose absorption via interaction with membrane abundance of SGLT1 in mice. SGLT1 is a protein involved in the process of transporting glucose and galactose across the membrane of the intestinal epithelial cells, so the sugar can be absorbed and used by the body.

The effect of regular ingestion of Gum Arabic on body mass index and body fat percentage on healthy adult females is significant reduction in body mass index (BMI) and body fat percentage. Gum Arabic readily dissolves in water to form solutions characterized by low viscosity. This feature makes it useful in various applications as an emulsifier, thickening reagent, flavor stabilizer and rheology modifier in liquid drinks. It is compatible with other natural dietary water-soluble fibers such as Aloe vera, Locust bean (Carob) gum, Pectin, Xanthan gum, hydroxy cellulose and alginates of sodium.

The compatibility feature of Gum Arabic makes it feasible to formulate specialty dietary fiber blends that also function as chelators against harmful metals such as Strontium, Barium, Chromium, Lead, Mercury, Cadmium, Arsenic and Aluminum in addition to the functions mentioned above. In the formulae of this invented line, Gum Arabic or any of its proprietary blends with other soluble fibers, will coexist with ionic and nonionic compounds, minerals, proteins, emulsifiers and any of the oils listed for this embodiment.

### Sweetener

Sweetener reagents are taken from botanical sources, organic or natural sugar, brown sugar, local honey, manuka honey, organic honey, xylitol, Stevia, Erythritol, natural unprocessed agave syrup and monk fruits. The used honey, in particular, must free from any of the USA known pesticides' categories and Clostridium botulinum bacteria that can cause infants Botulism.

An individual sweetener or a blend of sweeteners may be applied to attain the acceptable degree of sweetness on the brix scale. At 20 C 1.0 brix is equal to 1 gram of sugar added to 99 grams of water to make 100 grams of solution. The degree of brix of the beverage is adjusted to an acceptable range to suit coordinated with the nature and composition of the liquid drink line ingredients.

The claimed percentage of organic or natural sugar in any of the proprietary sweetener blend with other sweeteners listed for this embodiment is between 5% and 80%.

### Preservative

The Preservatives used in the formulae are all-natural food grade FDA approved such as: Sodium benzoate and Potassium Sorbates. However, in pursuit of presenting natural line of sport drink products, some essential oils known for its antimicrobial quality has been identified to aid in preserving the products against spoilage. These essential oils are white Thyme oil, Basil oil, Radish oil, Peppermint oil, Cinnamon oil, Capsicum oil and cloves oil. These oils may be used alone or in a claimed proprietary blend with the sodium or potassium sorbate salts.

The proprietary preservatives blends are able to inhibit the growth of microbes, extend the shelf life while maintaining the natural integrity of the embodiment. The level of the preservatives added individually or in a blend depends on factors related to the nature of the formula composition of each drink. The embodiment claimed percentage of sodium or potassium sorbate in the preservative blend containing essential oils shall be between 5% to 95% by weight. However, to avoid contamination all equipment and water content of the formula shall be sterilized.

### pH Controller

Hydrogen Ion Control Reagents: pH level of finished product shall be in the range of 6.0-8.0. Ingredients to adjust the pH level shall be FDA approved for use in the food and beverage industry, such as Citric acid, Sodium hydroxide, Citric acid, Sodium citrate, Carbonate of sodium, Carbonates of calcium, Sodium phosphate, Acetic acid and Sodium acetate, Lactic acid and Sodium lactate. Buffer system may be used to hold the pH level within the desired range.

### Emulsifier

Food Emulsifiers selected for the use in this product line are Carrageenan, Carboxymethyl cellulose, Polysorbates, Carboxymethyl Hydroxypropyl Guar Gum, Diacetyl tartaric acid ester of monoglyceride, Mustard, Soy lecithin, Sodium stearoyl lactylates, Sodium phosphates and Egg yolk. The use of an individual one of said food emulsifiers or in combination with others will be suitable to deliver the desired stability to the products of this invented line. The claimed percentage by weight of an emulsifier of any sport drink of the products' line is between 0.5% and 2.5% by weight.

### Rheology Modifier

The thickening reagents (rheology modifiers) that may be used in the manufacture of this line are Potato starch, Tapioca starch, Arrow root starch, Gelatin, Xanthan gum and Hydroxy cellulose compounds. The use of an individual one of said thickening reagents or in combination with others is expected to provide acceptable level of viscosity, stability and acceptable rheology. They may be added individually or in a proprietary blend at temperature between 50 C and 80 C with moderate agitation. The claimed percentage of arrow root starch in a thickener blend shall be between 0.5 and 7% by weight.

### Mineral

Mineral salts contemplated to be part of the formulae of this embodiment are Sodium Chloride, Sodium citrate, Sodium Carbonate, Calcium Chloride, Calcium Carbonate, Calcium citrate, Chlorides of Mg+2 or Magnesium chloride, Magnesium citrate, Potassium chloride, Potassium carbonate, potassium Iodide, Iron (III) Citrate (Ferric Citrate), Ferrous sulfate, Ferrous fumarate, Ferrous gluconate, Zinc citrate, Zinc chloride, Disodium phosphate, Dipotassium phosphate, Phosphatidylcholine and phosphatidylserine. They are added individually or in combination at a level of 100 to 900 ppm.

### Butters

The list of butters is to enhance the rheology and flavor of this beverage line are Shea butter, Mango butter, Cocoa butter, Ghee, Cupuacu butter, Kokum butter and Amish roll butter. They are, in some formulae combined with casein and food emulsifiers. They function as partial solubilizer and coupling. Butters are added individually or as a blend. The claimed percentage of Cocoa butter in a proprietary blend with other butters in the list shall be between 5% and 95% by weight.

### Protein

The list of Proteins selected for use in making this line includes Casein and Whey (Milk protein), Oat protein, Barley protein, Wheat protein, Albumen (egg white) and permalean protein. They are added individually or in a proprietary blend with other members on the list. The percentage of Casein or whey proteins in a proprietary blend of proteins with other members on the list shall be between 1% and 95% by weight.

### Trace Mineral

Trace minerals list selected for this embodiment, are also called microminerals. They are needed in nutrition but usually in tiny amounts. They are usually used in the formulae at a level between 100 to 1000 ppm. The trace minerals' list selected for the use of this beverage drink includes Boron compounds such as Boron citrate, Boron gluconate, Boron asparate, Boron glycinate and Calcium fructoborate. Other minerals are Cobalt, in the form of cobalamin (which forms an integral part of Vitamin 812), Copper, best in the form of chelated or citrated copper, Molybdenum glycinate, Cesium chloride and Selenium compounds such as Sodium selenite, Sodium selenate, Selenomethionine, Chromium chloride, Chromium nicotinate, Chromium picolinate, Manganese sulfate, Manganese gluconate, Manganese ascorbate and amino acid chelate of manganese. The trace minerals are added individually or in a proprietary blend with other minerals on the list. The amount of Boron citrate in a proprietary blend of trace mineral shall be between 25 ppm and 150 ppm.

### Embodiment

The method of manufacturing involves loading 80 C degree sterilized heated water into a sterilized closed head kettle. Loaded water is followed by moderate agitation and the addition of the designated number of preservatives and minerals. Then, sifting the dietary fiber Gum Arabic or the proprietary fiber blend to dissolve it to a clear solution at moderate high agitation. The sweetener(s) reagent is then added to dissolve to a clear solution.

The essential oils are separately combined together according to the designated essential oil proprietary blend. The essential oils' proprietary blend is loaded onto dairy fats of 18% minimum or combined with the suitable food emulsifier at 75 C then mixed for few minutes with gentle agitation. The emulsified essential oil mix is then added to the batch. The batch is mixed well at 80 C. The amount of milk is added and mix well. Apply homogenizer for 3 cycles then adjust the pH range to be between 6.5 and 8.5 using assigned pH control reagents.

Filter solution using 400 mesh filter cloth and apply cooling system gradually while blending at low moderate speed. Cool to ambient temperature then sample for QC and lab works. Any additional additives such as carbohydrates, proteins or fats prone to modify the rheology may be added at the end. The specific gravity is between 0.75-1.15 gm/cm3. This product is recommended to pack in coated aluminum pouches, waterproof cartons or Nalgene plastic bottles with Nitrogen gas blanket at the surface of the liquid.

The target audience of this product shall be all genders of healthy children beginning of age 5, youth, adult persons, elderly consumers, Athletes, dairy industry managers and nutritional research specialists of all geographic, psychographic, behavioral and demographic areas.

### Manufacturing Procedures

There are three manufacturing procedures disclosed for this line of products:

### Method I

Load the sterilized manufacturing kettle with the amount of water and warm up to 80 degree C., maintain temperature while moderately agitating.

Add the designated amount of food preservatives & minerals, then continue agitation until complete solubility.

Slowly sift amount of Gum Arabic through 200 mesh Sieve while agitating at a moderate speed to attain lump free clear solution.

Add the formula amount of sweetener reagent and continue agitation until clear solution.

Combine essential oils in the designated ratio and mix well at 50 deg C.

Combine essential oils' blended amount and dairy fats of minimum 18% or the suitable emulsifier and mix gently at 60-degree C. for homogeneous blend.

Add the essential oils/emulsifier blend to the batch and mix at moderate speed.

Add designated amount of milk and mix well.

Add additional additives such as carbohydrates, proteins and fats to modify rheology and mix well.

Apply homogenizer for 3 cycles.

Filter solution using 400 mesh screen or filter cloth then cool off batch to ambient temperature.

Sample for QC & lab works.

### Method II

Load the sterilized manufacturing kettle with the formula amount of water and warm up to 80 degree C., maintain temperature while gently agitating.

Add the formula amount of sweetener reagent and continue agitation until clear solution.

Add the designated amount of food preservative & minerals, then continue agitation until complete solubility.

Slowly sift amount of Gum Arabic fiber or the fiber blend through 200 mesh Sieve while agitating at a moderate speed to attain lump free clear low viscosity solution.

Combine essential oils in the designated ratio and mix well at 50 deg C.

Combine essential oils' blend and the dairy fats of minimum 18% or suitable emulsifier and mix gently at 60 degree C. for homogeneous blend.

Add amount of designated milk and mix well.

Add additional additives such as carbohydrates, proteins and fats to modify rheology and mix well.

Apply homogenizer for 3 cycles.

Filter solution using 400 mesh screen filter or filter cloth then cool off batch to ambient temperature.

Sample for QC & lab works.

### Method III

### Part A

Charge water to sterilized kettle a, turn agitator on to moderate speed while warming up to 80 deg C.

Add preservatives & minerals' amounts in the formula until completely dissolved while maintaining temperature at 80 C.

Add emulsifier then slowly sift amount of Gum Arabic through 200 mesh Sieve while agitating at a moderate speed to attain lump free clear solution. Maintain batch agitation at 80 C.

### Part B

Charge amount of milk to sterilized kettle b, warm up to 80 deg C. while gently agitating.

Add designated amount of sweetener and mix for complete solubility.

Combine contents of kettle b to Kettle a while still at 80 deg C. and mix well at moderate high speed.

Combine essential oils in the designated ratio and mix well at 50 deg C.

Combine essential oils' blend amount and amount of dairy fat of minimum 18% and mix gently at 600-degree C. for homogeneous blend.

Add the essential oils/dairy fat blend to the batch and mix well at moderate speed.

Add carbohydrates, proteins and fats to modify rheology.

Apply homogenizer for 3 cycles.

Filter solution using 400 mesh screen filter or filter cloth.

Cool off batch to ambient temperature.

Sample for QC & lab works.

### Formula Composition

The general formulae of this line of products comprises the following basic category of ingredients: Water, Dietary fiber, Essential oils' blend and Milk.

The water is purified, noncarbonated, sterilized and validated by microbial test. The pH ranges from 6 to 8.5. The water type shall be main or spring. Total traces of ions such as K+, Na+, Mg2+ and Ca2+ may not exceed 10000 parts per million total. The claimed percentage composition of water is between 5% and 25% by weight.

The dietary fiber of the general formula of the line is Gum Arabic and its blends. It is used alone or mixed with other compatible water-soluble dietary fibers such as Aloe vera, Locust bean (Carob) gum, Pectin, Xanthan gum, Hydroxy cellulose and alginate of sodium. The percentage of Gum Arabic used alone or fiber blend in the formula is between 0.5% and 3.5% by weight. In the specialty blends with other fibers, Gum Arabic constitutes 5% to 50% by weight. The percentage of the dietary specialty blends in the products' formulae vary between 0.5% and 4.5% by weight.

The main essential oils used in this line are sweet fennel seed and Star anis oils. The fennel seed oil, if added alone, constitutes between 0.10% and 0.60% by weight, whereas when it is blended with Star anis oil or any other member of the essential oils listed in the embodiment is between 10% and 80% by weight.

Milk shall be sterilized non-GMO organic or natural, whole or skimmed coming from grass fed farm animals and free from antibiotics or hormones. The percentage of milk in the formulae is between 50% and 95% by weight.

The sweetener sugar is organic or natural. The percentage of the disaccharide is between 2% and 9% by weight. Sweeteners listed in the detailed description may be mixed with the disaccharide in a specialty blend. The percentage of the organic or natural sucrose in the sweetener blend is between 5% and 75% by weight.

Preservatives are all food grade FDA approved such as Sodium benzoate, Potassium Sorbates. In addition, some essential oils known as microbial inhibitors are claimed and may be used such as White Thyme oil, White fir oil, Watercress oil, Basil oil, Radish oil, Peppermint oil, Cinnamon oil, Cumin seed oil, Ginger oil, Clary sage oil, Master seed oil, Arugula oil, Cardamon oil, Lemongrass oil, Turmeric oil, Tea tree oil, CBD oil, Oregano oil, White fir oil, Frankin cense oil, Maca oil, Lavender oil, Capsicum· oil, Mustard oil, Moringa oil, Argan oil, Carrot oil, Garlic oil, Onion oil and Cloves oil.

Some of the essential oils exhibit high activity against Gram-Positive and Gram-negative bacteria and some such as Maca oil exhibit Anthelmintic properties. Thus, may be used as preservatives, microbial inhibitors or as antibiotics, in the products' formulae, individually or in proprietary blends with preservatives' salts of Sodium and Potassium. The percentage of the antimicrobial essential oil in a blend with Sodium benzoate or Potassium sorbate vary between 5% to 75% by weight.

Minerals for this embodiment include Sodium Chloride, Sodium citrate, Sodium Carbonate, Calcium Chloride, Calcium Carbonate, Calcium citrate, Magnesium chloride, Magnesium citrate, Potassium chloride, Potassium carbonate, Potassium Iodide, Iron (III) Citrate (Ferric Citrate), Ferrous sulfate, Ferrous fumerate, Ferrous gluconate, Zinc citrate, Zinc chloride, Disodium phosphate, dipotassium phosphate, Phosphatidylcholine and phosphatidylserine.

The pH control reagents used for this embodiment are Sodium Hydroxide, Potassium hydroxides, Sodium carbonate, Potassium carbonate, Acetic acid, Magnesium citrate & Magnesium sulfates, Sodium acetate, Lactic acid, Sodium lactate, and Malic acid.

Emulsifiers selected for use in this product line are Carrageenan (a sea weed, sulfated polysaccharide also, called Irish moss), Carboxymethyl cellulose, Polysorbates, Carboxymethyl Hydroxypropyl Guar Gum, Diacetyl tartaric acid ester of monoglyceride, Mustard, Soy lecithin, Sodium stearoyl lactylates, Sodium phosphates and Egg yolk. The use of an individual one of said food emulsifiers or in combination with others will be suitable to deliver the desired stability to the line of this product. The percentage of Polysorbate in a blend with an emulsifier on the list shall be 35% by weight or less.

The list of Proteins selected for use in making this line includes Casein and Whey (Milk protein), Oat protein, Barley protein, Wheat protein, Albumen (egg white) and Permalean protein. They are added individually or in a proprietary blend with other members on the list. The percentage of Casein or whey proteins in a proprietary blend of proteins with other members on the list shall be between 1% and 95% by weight.

Trace minerals selected for this embodiment are also called microminerals. They are needed in nutrition but usually in tiny amounts. They are usually used in the formulae at a level between 10 to 100 ppm. The trace minerals selected for the use of this beverage drink includes Boron compounds such as Boron citrate, Boron gluconate, Boron asparate, Boron glycinate and Calcium fructoborate. Other minerals are Cobalt, in the form of cobalamin (which forms an integral part of Vitamin 812), Copper, best in the form of chelated or citrated copper, Molybdenum glycinate, Cesium chloride and Selenium compounds such as Sodium selenite, Sodium selenate, Selenomethionine, Chromium chloride, Chromium nicotinate, Chromium picolinate, Manganese sulfate, Manganese gluconate, Manganese ascorbate and amino acid chelate of manganese. The trace minerals are added individually or in a proprietary blend with other minerals on the list. Any member of the trace minerals on the list shall vary between 10 and 100 ppm with a total combined trace mineral per formula not to exceed 1000 ppm per formula.

The thickening reagents (rheology modifiers) that may be used in the manufacture of this line are Potato starch, Tapioca starch, Arrow root starch, Gelatin, Xanthan gum and Hydroxy cellulose compounds. The use of an individual one of said thickening reagents or in combination with others is expected to provide acceptable level of viscosity, stability and acceptable rheology. They may be added individually or in a proprietary blend at temperature between 50 C and 80 C with moderate agitation. The claimed percentage of arrow root starch in a thickener blend shall be between 0.5% and 7% by weight.

1. A sport drink comprising ingredients of water, preservative, mineral, trace mineral, dietary fiber, milk, sweetener, essential oil, emulsifier, at least 18% dairy fat, rheology modifier, vitamin, and protein.

2-25. (canceled)

26. The sport drink of claim 1, wherein a weight percent of the ingredients comprises:
water from 5% to 25%,
preservative from 0.01% to 0.19%,
mineral from 0.01% to 0.09%,
trace mineral from 0.001% to 0.009%,
dietary fiber from 0.5% to 3.5%,
milk from 55% to 95%,
sweetener from 2% to 9%,
essential oil from 0.15% to 0.55%,
emulsifier from 0.5% to 1.5%,
rheology modifier from 0.25% to 1.75%,
vitamin from 0.05% to 0.09%, and
protein from 0.1% to 1.9%.

27. The sport drink of claim 1, wherein a ratio of water to milk is from 1:2.2 to 1:19.

28. The sport drink of claim 1, wherein a weight percent of the essential oil is from 0.10% to 0.60%.

29. The sport drink of claim 1, wherein a weight percent of sweet fennel oil in the essential oil is from 10% to 80%.

30. The sport drink of claim 1, wherein a weight percent of sucrose in the sweetener is from 5% to 75%.

31. The sport drink of claim 1, wherein the essential oil has antimicrobial properties.

32. The sport drink of claim 1, wherein the preservative comprises at least one of sodium benzoate and potassium sorbate, and a weight percent of the preservative to essential oil is from 5% to 75%.

33. The sport drink of claim 1, wherein a weight percent of polysorbate in the emulsifier is no more than 35%.

34. The sport drink of claim 1, wherein a weight percent of at least one of casein and whey in the protein is from 1% to 95%.

35. The sport drink of claim 1, wherein a concentration of any one trace mineral in the sport drink is from 10 ppm to 100 ppm, a concentration of all the trace mineral in the sport rink is no more than 1000 ppm.

36. The sport drink of claim 1, wherein a weight percent of arrow root starch thickener in the rheology modifier is from 1% to 95%.
